(19) United States
(12) Patent Application Publication
(10) Pub. No.: US 2008/0188952 A1
(43) Pub. Date: Aug. 7, 2008

(54) PRE-POSITIONABLE PROSTHETIC HAND

(75) Inventors: Bradley Delton Veatch, Westminster, CO (US); Joseph David Scott, Centennial, CO (US)

Correspondence Address: SHERIDAN ROSS PC, 1560 BROADWAY, SUITE 1200, DENVER, CO, 80202, US

(73) Assignee: ADA TECHNOLOGIES, INC., Littleton, CO (US)

(21) Appl. No.: 12/026,454

(22) Filed: Feb. 5, 2008

Related U.S. Application Data
(60) Provisional application No. 60/888,234, filed on Feb. 5, 2007.

(51) Int. Cl. A61F 2/66; A61F 2/54

(52) U.S. Cl. 623/57; 623/65

(57) ABSTRACT

The present invention is directed to a pre-positionable prosthetic hand, including one or more pre-positionable joints. Pre-positionable joints in accordance with embodiments of the present invention include a multi-axial thumb joint, a metacarpal-phalangeal joint and a wrist joint. Additionally, the present invention may include a number of pre-positionable phalanges or fingers having a segmented construction, which allows them to be placed in various positions. The present invention may be used in connection with a pentadactyl glove and may be positioned in such griping positions as: a key or lateral grip, a hook grip, a cylindrical grip, a spherical grip and a tip-pinch grip.

### CROSS REFERENCE TO RELATED APPLICATIONS

The present application claims the benefit of U.S. Provisional Application No. 60/888,234, filed on Feb. 5, 2007, the entire contents of which are incorporated herein by reference in their entirety.

### STATEMENT REGARDING FEDERALLY SPONSORED RESEARCH OR DEVELOPMENT

This invention was not conceived under outside or external funding. Since its inception, however, an SBIR grant was applied for and received from the National Institute of Child Health and Human Development (NICHD), Grant No. 1R43HD054091-01 to reduce the invention to practice and validate its technical and economic validity.

### FIELD

The present application relates to an artificial or prosthetic hand having one or more of a pre-positionable thumb, finger, metacarpal-phalangeal and wrist joint.

### BACKGROUND

The following text should not be construed as an admission of knowledge in the prior art. Furthermore, citation or identification of any document in this application is not an admission that such document is available as prior art to the present invention, or that any reference forms a part of the common general knowledge in the art.

Individuals with upper extremity amputations may be provided with prosthetics, which can achieve various levels of functionality. Pre-positionable prosthetics typically have no motorized components and are designed to be manually moved into various positions by a user or wearer. Globally, many individuals having upper extremity amputations typically have only a single amputation. These individuals can be considered candidates for pre-positionable prosthetics. If a pre-positionable prosthetic hand is to be used, the individual may use his or her remaining hand to position the artificial hand.

Prosthetics, whether pre-positionable or not, can be quite costly to design and manufacture. As a result, prosthetics can be prohibitively expensive for those who need them. Moreover, in particular cultures, individuals having artificial or prosthetic limbs may face prejudices based on the appearance of the prosthetic. For example, prosthetic hands are available, which include mechanical hooks from gasping. While these prosthetics may be highly functional, they may be rejected by individuals who fear they will present a too mechanical appearance. On the other extreme, prosthetics with a highly realistic appearance may be rejected by those who fear that they will present an appearance of having been taken from a cadaver.

Accordingly, it would be desirable to have an artificial or prosthetic hand that can meet the functional, economic, and cosmetic needs of a wide variety of people. In that regard, the present invention is directed to a functional low-cost prosthetic having a generally acceptable appearance of “cosmesis.”

Nothing herein is to be construed as an admission that the present invention is not entitled to antedate a patent, publication or invention by another by virtue of prior invention.

### SUMMARY

It is to be understood that the present invention includes a variety of different versions or embodiments, and this Summary is not meant to be limiting or all-inclusive. This Summary provides some general descriptions of some of the embodiments, but may also include some more specific descriptions of certain embodiments.

The present invention is directed to a pre-positionable prosthetic hand adapted to be used by individuals having one or more upper extremity amputations. The prosthetic or artificial hand includes a number of pre-positionable joints. Pre-positionable joints in accordance with embodiments of the present invention include a multi-axial thumb joint, a metacarpal-phalangeal joint and a wrist joint. Additionally, the present invention may include a number of pre-positionable phalanges or fingers having a segmented construction, which allows them to be placed in various positions.

In at least one embodiment, the multi-axis thumb joint includes an attachment member, such as U-bolt, that interconnects a thumb member to a palm plate. The thumb may move along the attachment member to achieve neutral and pronated positions. Additionally, the thumb may move around the attachment member to achieve open and closed positions. The thumb joint additionally includes a thumb spring, which applies a tensile force to a fabric cord, which is, in turn, interconnected to the thumb. The tension in the fabric cord exerts a force on the thumb, which urges the thumb in a rotational movement around the attachment member towards the palm plate. The fabric cord is interconnected to the thumb through a bushing, which travels in a bushing track associated with the thumb. As the thumb rotates about the attachment member, the bushing travels within the bushing track to enable the fabric cord to exert a force on the thumb at a variety of positions.

In at least one embodiment, the metacarpal-phalangeal joint includes a metacarpal section, which is lockable in a number of angular positions with respect to the palm plate. In that regard, the metacarpal section may include a fixed connection to an axle. The axle, in turn, is rotatably mounted to the palm plate. Additionally, the axle is translatable within two mounting points between locked and unlocked positions. In the unlocked position, the axle, and thus the metacarpal section, is free to rotate with respect to the palm plate. In the locked position, the metacarpal section is locked in a number of discrete positions with respect to the palm plate.

In at least one embodiment, the wrist joint includes a wrist core, which is fixedly attached to the palm plate, and a wrist cup, which is fixedly attached to a base member or attachment boss. Additionally, the wrist core and attachment boss include a fixed connection to a bead, which is operatively associated with a cavity in the wrist core. The wrist core is adapted to move around the bead in a movement guided by the wrist core. In this way, the palm plate is able to articulate with the base of the base member or attachment boss.

In accordance with embodiments of the present invention, the prosthetic or artificial hand is designed to be worn inside of a pentadactyl glove. In this way, the prosthetic presents the appearance simply of a gloved hand. As a result, the user avoids the problems associated with having either a too mechanical or too life-like hand.

In accordance with embodiments of the present invention, the artificial hand is positionable in a variety of positions or grips. Grip positions in accordance with the present invention include in a key or lateral grip, a hook grip, a cylindrical grip, a spherical grip and a tip-pinch grip.

As used herein, “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Various embodiments of the present invention are set forth in the attached figures and in the detailed description of the invention as provided herein and as embodied by the claims. It should be understood, however, that this Summary does not contain all of the aspects and embodiments of the present invention, is not meant to be limiting or restrictive in any manner, and that the invention as disclosed herein is and will be understood by those of ordinary skill in the art to encompass obvious improvements and modifications thereto.

Additional advantages of the present invention will become readily apparent from the following discussion, particularly when taken together with the accompanying drawings.

### BRIEF DESCRIPTION OF THE DRAWINGS

FIG. 1A is a side elevation view of an artificial hand, in accordance with embodiments of the present invention;

FIG. 1B is a perspective view of the artificial hand show in FIG. 1A;

FIG. 1C is a view of the artificial hand shown in FIG. 1A, looking at the palm of the hand;

FIG. 1D is a view of the artificial hand shown in FIG. 1A, looking at the fingers of the hand;

FIG. 2A is reverse perspective view of the artificial hand shown in FIG. 1A;

FIG. 2B is enlarged view of a portion of the artificial hand shown in FIG. 2A;

FIGS. 3A-3C are perspective views showing various positions of the artificial hand shown in FIG. 1A;

FIGS. 4A-4C are perspective views showing other various positions of the artificial hand shown in FIG. 1A;

FIG. 5 is an exploded view of a metacarpal-phalangeal joint, in accordance with embodiments of the present invention;

FIGS. 6A-6D are perspective views illustrating a movement of the metacarpal-phalangeal joint shown in FIG. 5;

FIGS. 7A-7D are side elevation views showing various positions of the metacarpal-phalangeal joint shown in FIG. 5;

FIG. 8 is an exploded view of the artificial hand shown in FIG. 1A;

FIG. 9 is a cross sectional view of a wrist joint in accordance with embodiments of the present invention;

FIGS. 10A-10B are perspective views illustrating a movement of the wrist joint shown in FIG. 9;

FIG. 11 is perspective view of the artificial hand shown in FIG. 1A, provided in association with a pentadactyl glove;

FIGS. 12A-12D are illustrations of positions of the artificial hand shown in FIG. 1A, the positions associated with a lateral grip;

FIG. 13 is an illustration of the artificial hand shown in FIG. 1A, positioned in a hook grip;

FIG. 14 is an illustration of the artificial hand shown in FIG. 1A, positioned in a cylindrical grip;

FIG. 15 is an illustration of the artificial hand shown in FIG. 1A, positioned in a spherical grip; and

FIG. 16 is an illustration of the artificial hand shown in FIG. 1A, positioned in a tip-pinch grip.

### DETAILED DESCRIPTION

The present invention is generally directed to a low cost prosthetic device for use by persons with upper extremity amputations. The prosthetic device is a terminal device, or artificial hand, which includes a number of moveable joints that allow a user to position the hand in a number of grasping modes. The prosthetic hand is preferably used by a person having a single extremity amputation. Accordingly, the amputee may use their remaining hand to position the prosthetic hand in the different grasping modes. Alternatively, individuals that are dual amputees may also use the device, wherein an object or another prosthetic may be used to position the present invention in the different grasping modes.

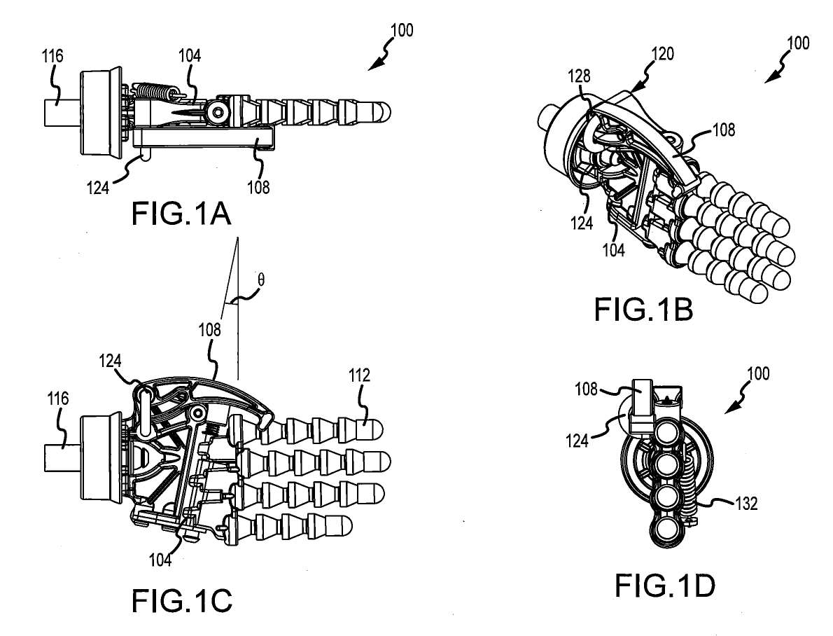

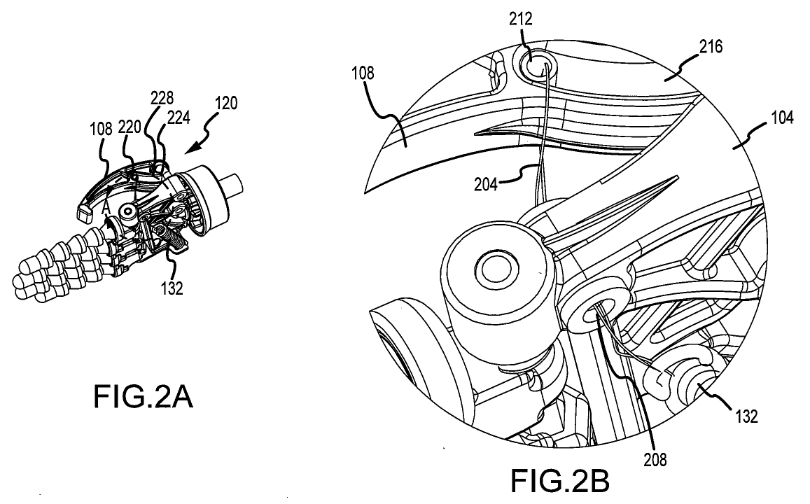

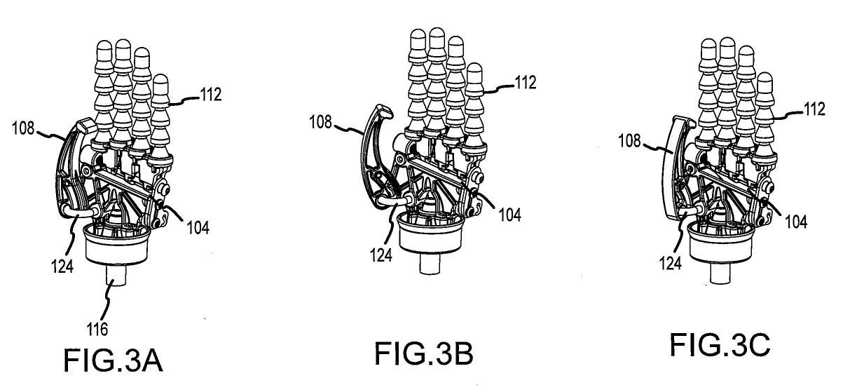

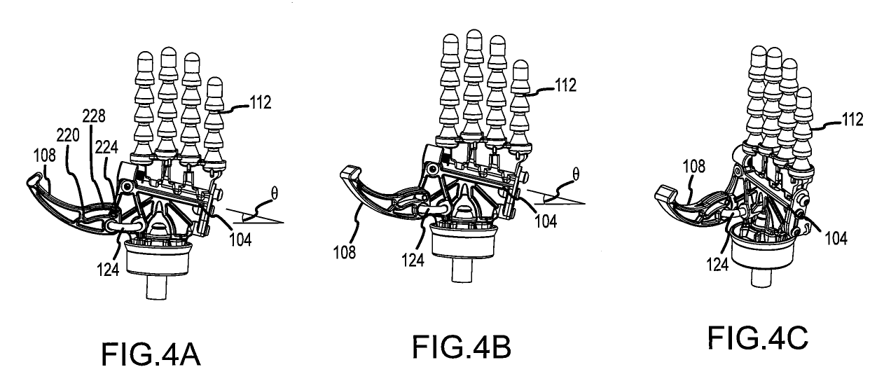

Turning initially to FIGS. 1A-1D, the artificial hand is generally identified by reference numeral 100. FIG. 1A shows the artificial hand 100 from a side elevation view. FIG. 1B shows the artificial hand 100 from a perspective view. FIG. 1C is a view of the artificial hand 100 showing the palm of the hand. FIG. 1D shows a view of the artificial hand 100 looking into the fingers of the hand. The artificial hand includes a palm plate 104, which articulates with a number of other components to provide the various grasping positions of the hand 100. Generally, the palm plate 104 articulates with a thumb 108 and with a number of phalanges 112 or fingers. Additionally, the artificial hand 100 may include a base member or attachment boss 116 that articulates with the palm plate.

Turning first to the thumb 108, the prosthetic device or artificial hand 100 may include a multi-axis thumb joint 120, which allows the thumb 108 to articulate with the palm plate 104. As best seen in FIG. 1B, the thumb joint 120 includes an attachment member 124, which in accordance with embodiments of the present invention is a U-bolt. The U-bolt 124 is received in a transverse hole 128, which is provided at the base of the thumb 108. The U-bolt 124 allows both a translational movement and a rotational movement of the thumb 108. In the translational movement, the thumb 108 may move along the U-bolt 124. In the rotational movement, the thumb 108 may rotate around the U-bolt 124. As best seen in FIG. 1D, the thumb joint 120 additionally includes a biasing member or thumb spring 132 disposed on the dorsal or back side of the artificial hand 100. The thumb spring 132 operates to at least restrict the rotational range of motion of the thumb 108.

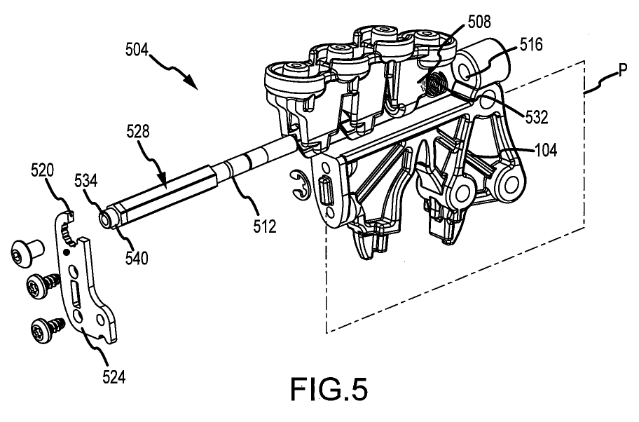

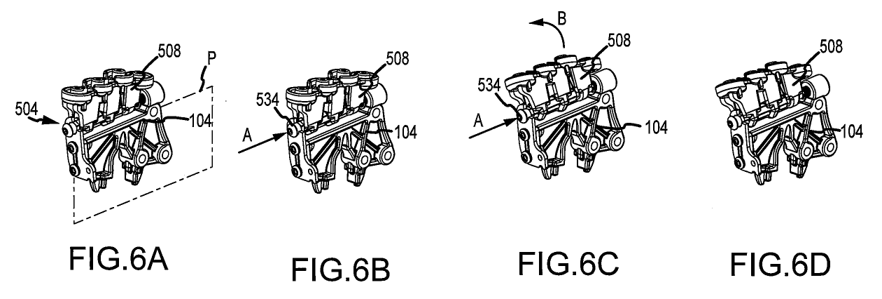

A more detailed understanding of the operation of the thumb joint can be had by an inspection of FIGS. 2A and 2B. FIG. 2A is reverse perspective view of the artificial hand 100, including the thumb joint 120. FIG. 2B is an enlargement of a portion of FIG. 2A, showing a close-up view of the thumb joint 120. As best seen in FIG. 2B, the thumb joint 120 includes a textile or fabric cord 204. The cord 204 may be made of materials such as, for example, cotton, polymer fibers or other textiles. In a separate aspect of the invention, the cord 214 may comprise one or more of KEVLAR® para-aramid, Vectran® liquid crystal polymer, NOMEX® heat resistant nylon, TEFLON® polytetraflouroethylene, SPECTRA® ultra high molecular weight polyethylene, as well as other materials sold under the brand names of TWARON®, ZYLON®, and TECHNORA®. In addition, other materials than those may be used to form the cord 214, such as, but not limited to plastic cord, metal cord, fabric cord, and combinations of any of the foregoing, including an outer layer of a first material and an inner tendon of a second material. The cord 204 is disposed through a transverse hole 208 in the palm plate 104. At one end, the cord 204 is attached to the thumb spring 132. At the other end, the cord 204 is connected to a bushing 212, which is moveable within a bushing track 216 associated with the thumb 108. The spring 132 exerts a tensile force on the cord 204, which in turn, acts on the bushing 212 to move the bushing 212 towards either the first end 220 or the second end 224 of the bushing track 216.

The end of the bushing track 216 to which the bushing 212 is moved by the tension in the cord 204 will depend on the location of the bushing 212 within the bushing track 216. In that regard, the bushing track 216 has an arcuate shape, which includes a neutral bias point 228. With the bushing 212 disposed at points between the first end 220 and the neutral point 228, the bushing 212 will be urged towards the first end 220 of the bushing track 216. With the bushing 212 disposed at points between the neutral point 228 and the second end 224 or with the bushing 212 at the second end, the bushing 212 will be urged towards the second end 224 of the bushing track 216.

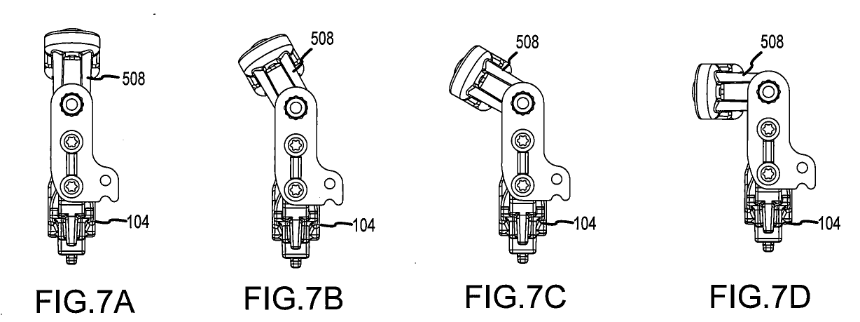

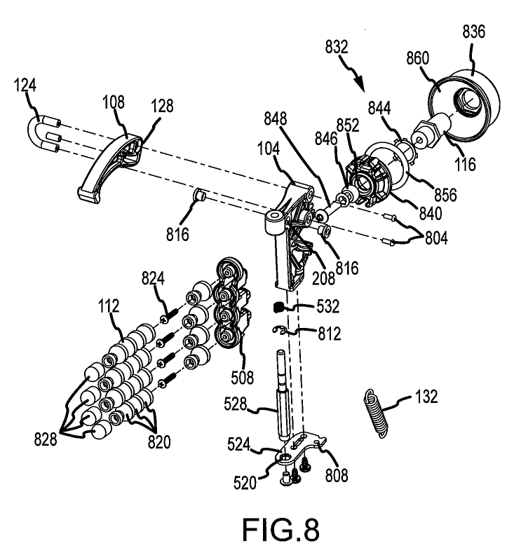

With the bushing 212 at a first end 220 of the bushing track 216, the thumb 108 is in an closed position. With the bushing 212 at a second end 224 of the bushing track 216, the thumb 108 is in a open position. As used herein, “open” and “closed” refer to the thumb's 108 rotational position about the U-bolt 124. As shown in FIGS. 3A-3C, the translational position of the thumb 108 along the U-bolt 124 may be varied, while the thumb 108 remains in a “closed” position. Similarly, as shown in FIGS. 4A-4C, the translational position of the thumb 108 along the U-bolt 124 may be varied, while the thumb remains in an “open” position.

Turning first to FIGS. 3A-3C, the thumb 108 is shown in a closed position at various translational positions along the U-bolt 124. In FIG. 3A, the thumb 108 is shown at its farthest extent of travel in one direction along the U-bolt 124. Here, the thumb is in a closed pronated position. In FIG. 3C, the thumb 108 is shown substantially at a center point along the U-bolt 124. Here, the thumb 108 is in a closed neutral position. In FIG. 3B, the thumb is in a closed semi-pronated position, between the closed pronated position shown in FIG. 3A and the closed neutral position shown in FIG. 3C. Opposite from the position shown FIG. 3A, the furthest extent of travel of the thumb 108 along the U-bolt 124 corresponds to an anatomically impossible supinated position (not shown). In particular, the normal human hand does not allow movement of the thumb much past the neutral position. Embodiments of the present invention may allow this anatomically impossible position for the thumb 108. Alternatively, a cuff or other restricting device, such as a detachably attachable sleeve or clip, may be provided on the U-bolt 124 to prevent movement of the thumb 108 into the supinated position. It is further within the scope of the present invention to use an arcuate-shaped member with lesser range of positioning locations than the U-bolt 124.

In the thumb positions shown in FIGS. 3A-3C, the thumb 108 is in a closed position, wherein the thumb 108 is rotated to its farthest extent towards the palm plate 104. In contrast, FIGS. 4A-4C, show the thumb 108 in an open position. In the open position, the thumb 108 is rotated about the U-bolt 124, away from the palm plate 104. In FIG. 4A, the thumb 108 is in an open pronated position. In FIG. 4B, the thumb 108 is in an open semi-pronated position. In FIG. 4C, the thumb 108 is in an open neutral position.

FIGS. 3A-3C and FIGS. 4A-4C illustrate the range of motion for the thumb 108. As illustrated therein, the thumb 108 is moveable in translational movement along the U-bolt 124 and in a rotational movement about the U-bolt 124. The thumb 108 may be placed by a user at a variety of positions along the U-bolt 124 limited by the points at which the U-bolt 124 is connected to the palm plate 104. With respect to the rotational movement of the thumb 108, the thumb 108 is biased by the thumb spring 132 towards either the open or the closed position. As mentioned above, the thumb spring 132 exerts a tensile force on the bushing 212 that urges the bushing towards either the first end 220 or second end 224 of the bushing track 212. In this way, the movement of the bushing 212 within the bushing track 216 causes the thumb 108 to rotate about the U-bolt 124. The user may rotate the thumb 108 through the rotational range of motion afforded by the cord 204. However, if the thumb 108 is released by the user, the tension in the cord 204 will operate to restore the thumb 108 to either the open or closed position. It is noted that the thumb 108 may be over rotated past the open position. Here, the bushing 212 remains in the second position, while thumb spring 132 extends to accommodate the over rotation. If the thumb 108 is then released, the tension in the cord 204 operates to restore the thumb 108 to the open position shown in FIGS. 4A-4B, while the bushing 212 remains in the second position.

Accordingly, the tension in the cord operates to maintain the thumb 108 in either the open or closed position, depending on the position of the thumb 108. Consider for example, the closed position of the thumb 108, as depicted in FIGS. 3A-3C. If the thumb 108 is rotated around the U-bolt 124, away from the palm plate 104, the thumb is thereby moved out of the closed position. Here, the bushing 212 moves away from the first position, along the bushing track 216. If the thumb 108 is released before the bushing 212 pass the neutral bias point 228, the tension in the cord 204 will cause the thumb 108 to be restored to closed position. If, however, the thumb 108 is rotated to a point where the bushing 212 passes the neutral bias point 228, release of the thumb 108 will result in the thumb being maintained in, or restored to the open position. Similarly, in moving between the open position (shown in FIGS. 4A-4C) and the closed position (shown FIG. 3A-3C), a user may rotate the thumb 108 towards the palm plate 104 to a point where the bushing 212 passes the neutral bias point 228. Here, if the thumb 108 is released, the tension in the cord 204 will cause the thumb 108 to be restored to the closed position. In accordance with embodiments of the present invention, the neutral bias point 228 is located at substantially the center of the bushing track 216. In accordance with alternative embodiments of the present invention, the neutral bias point 228 is located proximate to either the first end 220 or the second end 224 of the bushing track 216.

Additionally, it is noted that the tension in cord 204 allows the thumb to exert pressure on an object that is placed between the thumb 108 and the palm plate 104. The pressure can be exerted by the thumb 108 in either the closed or the open position, and can be exerted with the thumb 108 at any position along the U-bolt 124.

In accordance with embodiments of the present invention, the thumb 108 includes an angled tip, which, in FIG. 4C is generally identified by reference numeral 404. The angled tip 404 may is adapted to aid the thumb 108 in gripping and holding objects. Additionally, the angled tip 404 may be used to guide thumb 108 along a grooved or angled surface.

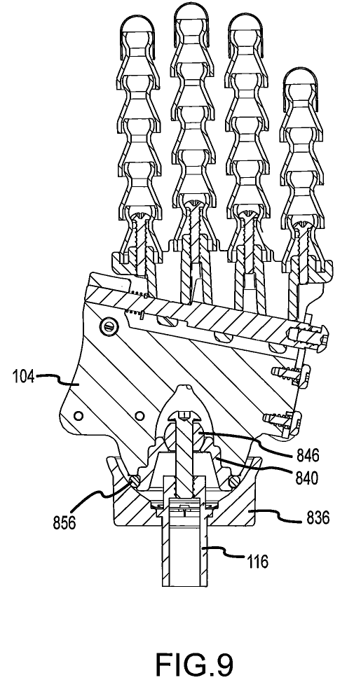

Turning next the phalanges 112 and their articulation relative to the palm plate 104, reference is made to FIG. 5, which shows an exploded view of a metacarpal-phalangeal joint 504, in accordance with embodiments of the present invention. The metacarpal-phalangeal joint 504 includes a metacarpal section 508, which rotatably attaches to the palm plate 104. In particular, the metacarpal section 508 is fixedly attached to an axle 512, which, in turn, is rotatably attached to the palm plate 104 at a first mounting point 516 and at a second mounting point 520. In accordance with embodiments of the present invention, the first mounting point 516 is a hole associated with palm plate 104. A mounting plate 524, which is attached to the palm plate 104 may include a hole that serves as the second mounting point 520.

The axle 512 includes an enlarged portion 528 having and N-sided polygon shaped cross-section. By way of example and not limitation, the enlarged portion 528 may have a hexagonal cross-section. The enlarged portion 528 is operatively associated with an M-sided hole associated with the second mounting point 520. By way of example and not limitation, the mounting point 520 shown in FIG. 5 includes a 12-pointed hole. Accordingly, the enlarged portion 528 can sit in the second mounting point 520 only at particular angular orientations. In the exemplary embodiment shown in FIG. 5, hexagonal enlarged portion 528 affords the metacarpal section approximately seven discrete positions between −90 and +90 degrees with respect to a plane P defined by the palm plate 104.

In seating the enlarged portion 528 in the second mounting point 520, both the first mounting point 516 and the second mounting point 520 allow translational movement of the axle 512. In that regard, the axle 512 includes an axle spring 532, which urges the axle 512 towards the second mounting point 520. Here, the user may press on an axle end 534 to move the axle 512 out of its seating in the second mounting point 520 against the action of the axle spring 532. It is noted that the axle end 534 includes an un-enlarged portion 540. When the user presses on the axle end 534, the un-enlarged portion 540 is located within the 12-pointed hole or second mounting point 520. Here, the diameter of the un-enlarged portion 540 is not large enough to engage the sides of the second-mounting point 520. Accordingly, the axle 512 is unseated. With the axle 512 unseated, the metacarpal section 508 is in an unlocked position, free to rotate with respect to the palm plate 104.

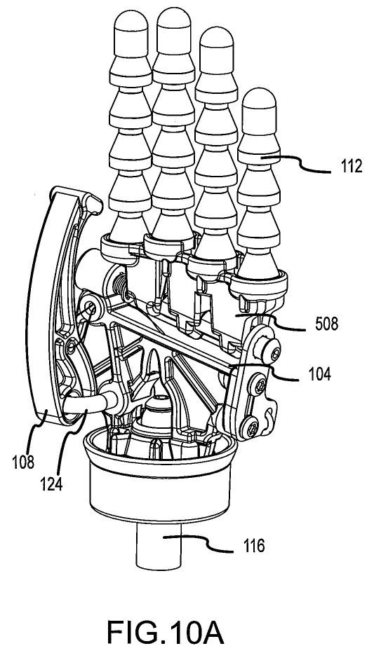

The positioning of the metacarpal-phalangeal joint 504 is illustrated in FIGS. 6A-6D. In FIG. 6A, the metacarpal-phalangeal joint 504 is shown in a first position, with the metacarpal section 508 disposed at a 0 degree angle with respect to the plane P defined by the palm plate 104. Here, the axle 512 is seated in the second mounting point 520 and, accordingly, the metacarpal section 508 is locked in place. In FIG. 6B, a user presses on the axle end 534, as indicated by the arrow A. As a result, the axle 512 moves against the action of the axle spring 532, to thereby unseat the enlarged portion 528 from the second mounting point 520. Here, the metacarpal section 508 is free to rotate with respect to the palm plate 104. In FIG. 6C, a user maintains the pressure on the axle end 534 (arrow A) and rotates the metacarpal section 508 out of plane P, as indicated by the arrow B. Here, the metacarpal section 508 is rotated into a position, which is approximately +30 degrees with respect to the palm plate 104. In FIG. 6D, the user releases the pressure on the axle end 534 and, accordingly, the axle 512 moves into a seated position in the second mounting point 520 under the action of the axle spring 512. Here, the axle 512, and thus the metacarpal section 508, are locked in place.

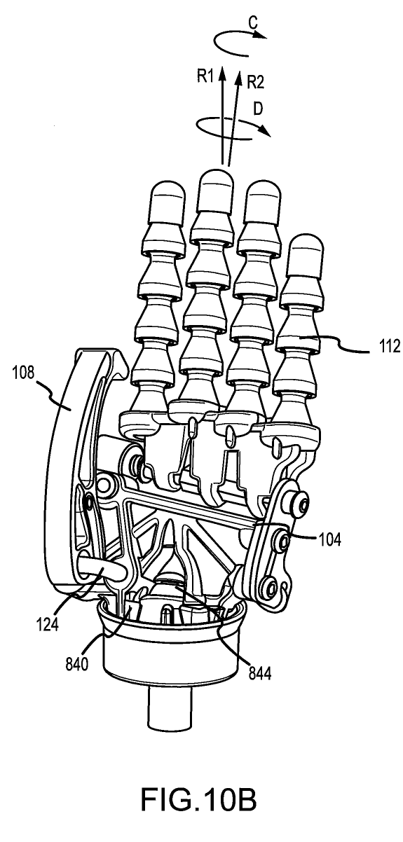

FIGS. 7A-7D are side elevation views of the metacarpal-phalangeal joint 504, which show the metacarpal section 508 disposed at various angles with respect to the palm plate 104. In FIGS. 7A-7D the axle 512 is seated in the second mounting point 520, and thus the metacarpal section 508 is locked in place. FIGS. 7A-7D illustrate the positions of metacarpal section 508 between 0 degrees and +90 degrees with respect to the palm plate 104, which are afforded by the hexagonal enlarged portion 528. In particular, FIG. 7A shows the metacarpal section 508 disposed at a 0 degree angle with respect to the palm plate 104 (also shown in FIGS. 6A and 6B). FIG. 7B shows the metacarpal section 508 disposed at a 30 degree angle with respect to the palm plate 104 (also shown in FIGS. 6C and 6D). FIG. 7C shows the metacarpal section 508 disposed at a 60 degree angle with respect to the palm plate 104. FIG. 7D shows the metacarpal section 508 disposed at a 90 degree angle with respect to the palm plate 104. In the embodiment of the present invention shown in FIGS. 7A-7D, the axle 512 will not seat in the second mounting point 520 with metacarpal section disposed at intermediate angles, which are between those afforded by the hexagonal enlarged portion 528. It is noted however, that locked positions at different angles are achievable with different configurations of the enlarged portion 528 and second mounting point 520. For example, an 8-sided or 12-sided enlarged portion 528 may used to achieve different locked positions. Any number of sides of the enlarged portion 528 and any number of sides for the second mounting point 520 are considered within the scope of the invention.

In accordance with embodiments of the present invention, the axle 512 is mounted at an angle θ, such as approximately a 12 degree angle relative to the base of the hand 100. This mounting angle can be seen in, for example, FIG. 1C and FIG. 4B. The 12 degree angle is based on the shape of the human hand. In particular, in the human hand the fingers meet the palm in at a line that slopes downward when the hand is held upright. This natural angle of the hand facilitates gripping objects when the hand is rotated into a vertical position. Modifications of angle θ are within the scope of the present invention.

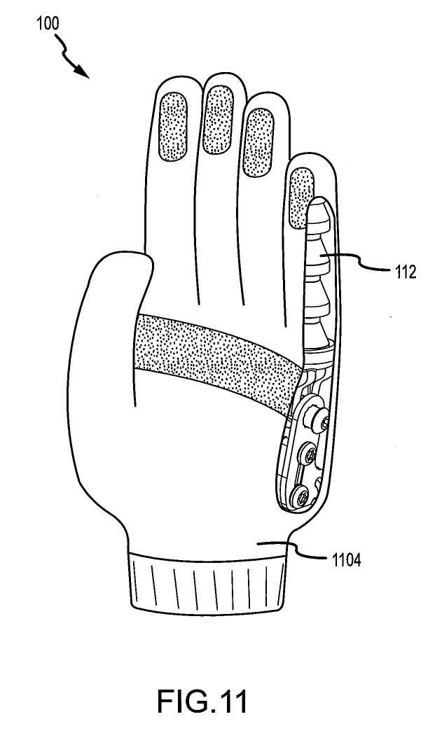

Turning now to FIG. 8, an exploded view of the artificial hand 100 is shown. FIG. 8 shows a detailed view of the interconnection between the components of the artificial hand 100. As can be seen in FIG. 8, the U-bolt 124 is received through the transverse hole 128 in the base of the thumb 108 and attached to palm plate 104 at both ends of the U-bolt 124. Pins 804 secure the ends of the U-bolt 124 to the palm plate 104. As described above, the mounting plate 524 attaches to an end of the palm plate 104 and provides the second mounting point 520 for the axle 512. Additionally, it is noted that the mounting plate 524 may include an attachment point 808 for an end of the thumb spring 132. As can be seen in FIG. 8, the axle spring 532 may be provided in association with a washer 812, which provides an engagement surface on which the axle spring 532 may act. As also can be seen in FIG. 8, the transverse hole 208 in the palm plate 104 may include one or more eyelets 816. The eyelets 816 may be made of polished ceramic to provide a smooth engagement surface for the cord 204 to contact as the cord 204 passes through the transverse hole 208.

FIG. 8 includes an exploded view of the phalanges 112 and the connection between the phalanges 112 and the metacarpal section 508. As can be seen, the phalanges 112 may include a number of interconnected segments 820. A particular phalange 112 may bend to a certain degree at each point where adjacent segments 820 meet. In this way, a phalange may be bent in a manner that approximates the range of motion of a human finger. As an alternative to the interconnected segments 820, the present invention may include a smaller number of joints disposed at locations that correspond to the knuckles and joints of the human hand. In the embodiment shown FIG. 8, the phalanges 112 may be interconnected to the metacarpal section 508 by screws 824 located internally to the phalanges 112. Additionally, the ends of phalanges 112 may include caps 828 that provide a seal, which prevents water or other fluids from entering the phalanges 112.

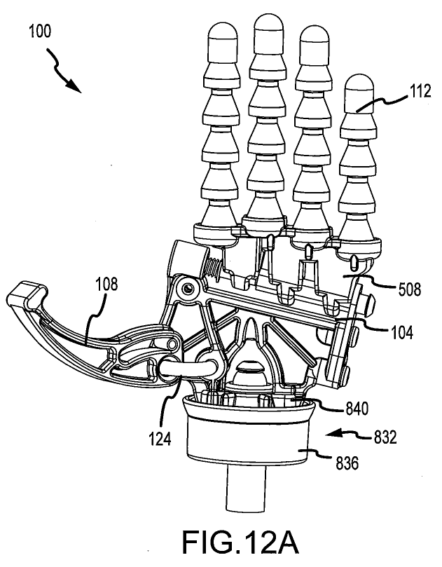

As mentioned above, the artificial hand 100 includes a base member or attachment boss 116. The attachment boss 116 is adapted to be received in or otherwise operatively associated with a mounting component (not shown) disposed on the extremity of the amputee at the point where the amputation occurred. In FIG. 8, the attachment boss 116 is shown in an exploded view along with components of a wrist joint 832, in accordance with embodiments of the present invention. The wrist joint 832 allows the palm plate 104 to move with respect to the attachment boss 116. The wrist joint 832 includes a wrist cup 836 fixedly attached to the attachment boss 116, and a wrist core 840 fixedly attached to the palm plate 104. A spring retainer 844 holds the attachment boss 116 in place with respect to the wrist cup 836. When assembled, the wrist core 840 is bonded to the palm plate 104. Alternatively, the palm plate 104 and the wrist core 840 may be formed together as one integral part (e.g., an injection molded part). The wrist core 840 is provided in association with a bead 846, which is fixed in place with respect to the attachment boss 116 and the wrist cup 836. In particular, a screw 848 is threaded through the bead 846 and received by an end of the attachment boss 116. The bead 846 sits in a center cavity 852 associated with the wrist core 840. The wrist core 840, in turn, sits in the wrist cup 836. The wrist core 840 may include an o-ring 856 disposed on a rim of the wrist core 840. As the wrist core 840 moves within the wrist cup 836, the o-ring 856 engages the inner surface 860 of the wrist cup 836. In particular, the wrist core 840 rotates around the bead 846 in a range of motion guided by the wrist cup 836. FIG. 9 shows a cross sectional view of the assembled wrist joint 832.

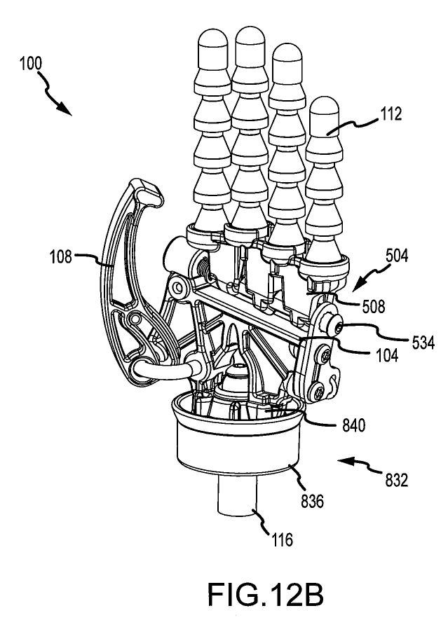

The movement of the wrist joint 832 is illustrated in FIGS. 10A-10B. In FIG. 10A, the palm plate 104 is aligned with respect to the attachment boss 116. In FIG. 10B, the palm plate 104 and the wrist core 840 are moved about the bead 846 into a position in which the axis R2 of the wrist core 840 is angled with respect to the axis R1 of the wrist cup 836. As indicated by arrow C, the palm plate 104 and the wrist core 840 may be rotated around the axis R2 of the wrist core through 360 degrees. Additionally, as indicated by arrow D, the palm plate 104 and wrist core 840 may move in a 360 degree “precessional” movement about the center axis R1 of the wrist cup 836.

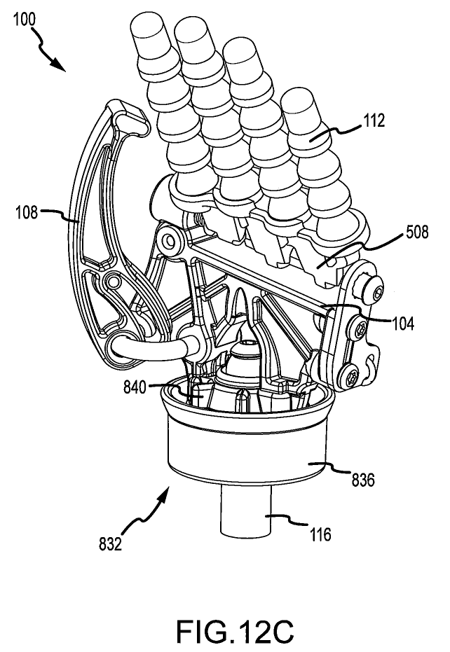

Referring now to FIG. 11, the artificial hand 100 may be provided in association with a pentadactyl glove 1104. A glove 1104 may be worn over the artificial hand 100 in order to present a more acceptable appearance. In particular, the mechanical components of the artificial hand are hidden and the prosthetic device simply presents the appearance of a gloved hand. Any type of glove 1104 may be worn over the artificial hand, including leather gloves or gloves having specially designed gripping surfaces.

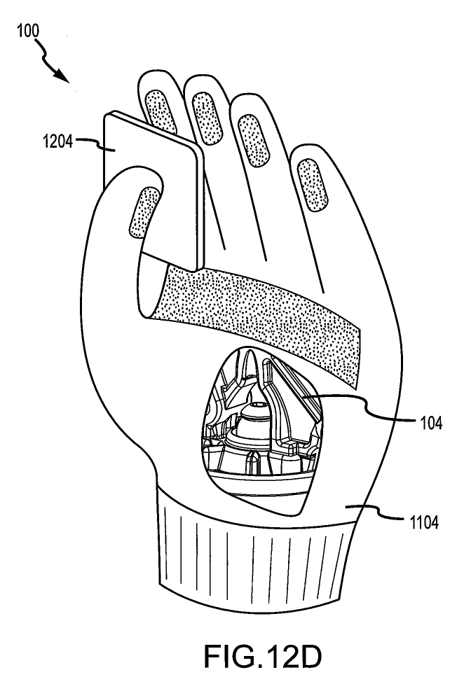

In use, the artificial hand 100 is adapted to achieve at least five distinct prehension patterns. In particular, the artificial hand 100 is positionable in a key or lateral grip, a hook grip, a cylindrical grip, a spherical grip and a tip-pinch grip. FIGS. 12A-12D show the manipulation or positioning of the artificial hand 100, which may be applied to achieve the lateral grip. As mentioned above, a glove 1104 is typically worn over the artificial hand 100, when the hand 100 is in use. However, in order to more clearly illustrate the positioning of the hand 100, the glove has been omitted from FIGS. 12A-12C. FIG. 12D, which shows the final position of the lateral grip, includes the glove 1104.

FIG. 12A shows an exemplary starting position in which the artificial hand 100 may be in prior to a user positioning the artificial hand 100 in the lateral grip. Here, the phalanges 112 are unbent. The metacarpal section 508 is disposed at a 0 degree angle with respect to the palm plate 104. The thumb 108 is disposed in a neutral and open position. The wrist joint 832 is oriented such that the axis of the wrist core 840 is aligned with the axis of the wrist cup 836.

FIG. 12B shows a first step or position into which the artificial hand 100 may be moved when the artificial hand 100 is positioned in the lateral grip. Here, the metacarpal-phalangeal joint 504 is manipulated to move the phalanges 112 and the metacarpal section 508 into position. In particular, the axle end 534 is pressed to move the axle 512 laterally against the action of axle spring 532, thereby unseating the axle 512 from the second mounting point 520. With the axle 512 unseated, the metacarpal section 508 is free to rotate with respect to the palm plate 104. Here, the user rotates the metacarpal section 508 approximately 60 degrees towards the palm plate 104 and releases the pressure on the axle end 534. As a result, the axle spring 532 causes a translational movement of the axle 512, which seats the axle 512 in the second mounting point 520. As a result, metacarpal-phalangeal joint 504 is locked in a position such that the metacarpal section 508 is disposed at approximately a 60 degree angle with respect to the palm plate 104.

FIG. 12C shows a second step or position into which the artificial hand 100 may be moved when the artificial hand 100 is positioned in the lateral grip. Here, the thumb joint 120 is manipulated to move the thumb 108 into position. In particular, the thumb 108 is moved along the U-bolt 124 from the neutral position to the pronated position. Additionally, the thumb 108 is rotated about the U-bolt 124 from the open position to the closed position. Here, the user rotates thumb 108 at least past the point where the bushing 212 moves past the neutral bias point 228 of the bushing track 216. With the bushing 212 past the neutral bias point 228, the tension in the cord 204 will cause thumb 108 to move into the closed position. In moving the artificial hand 100 into the lateral grip, other adjustments may be made. For example, the phalanges 112 may be bent towards the palm plate 104, or the wrist joint 832 may be manipulated to create a desirable angle for the palm plate 104 with respect to the attachment boss 116.

FIG. 12D shows the artificial hand 100 disposed in the key or lateral grip. Here, the metacarpal-phalangeal joint 504 is locked in a position such that the metacarpal section 508 is disposed at approximately a 60 degree angle with respect to the palm plate 104. Additionally, the thumb 108 is disposed in a closed and pronated position. In this position, the artificial hand 100 is used to grip a card key 1204. As can be appreciated, this orientation or position of the artificial hand 100 may be used to grip other, similarly shaped objects, such as for example, credit cards, keys, tickets and paper money. It is additionally noted that, in FIG. 12D, the phalanges 112 are bent in an angle towards the palm plate 104. While this orientation of the phalanges 112 may not necessarily aid in the griping of the key card 1204, it may present a more natural appearance when the artificial hand is placed in the lateral grip.

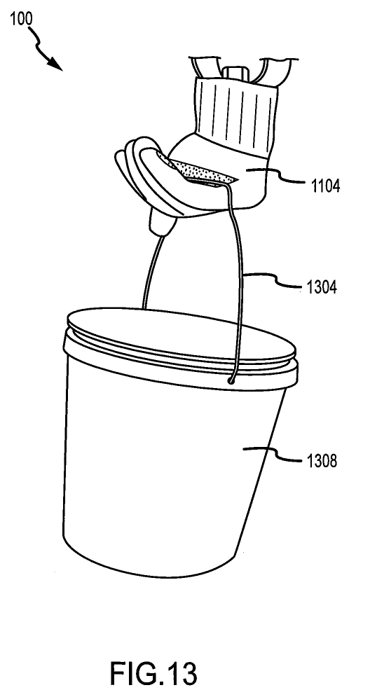

FIGS. 13-16 show additional prehension patterns which may be achieved by embodiments of the present invention. The artificial hand 100 may be moved from any position to the positions shown in FIGS. 13-16 through manipulations or movements of the various joints, as described herein. In FIGS. 13-16 the artificial hand 100 is shown in use, in combination with a glove 1104.

FIG. 13 shows the artificial hand 100 disposed in the hook grip. Here, the metacarpal-phalangeal joint 504 is locked in a position such that the metacarpal section 508 is disposed at approximately a 90 degree angle with respect to the palm plate 104. Additionally, the thumb 108 is disposed in a closed and pronated position. In this position, the artificial hand 100 is used to grip the handle 1304 of a bucket 1308. More specifically, the handle 1304 rests on the metacarpal section 508 and/or a portion of the phalanges 112, and the artificial hand 100 supports the weight of the bucket 1308. The phalanges 112 are bent in an angle towards the palm plate 104 to either present a natural appearance for the artificial hand 100, or to aid in preventing the handle 1304 from becoming dislodged from the artificial hand 100.

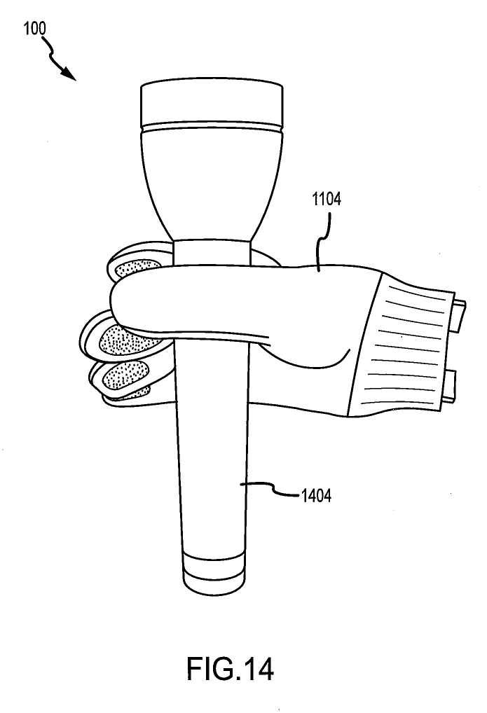

FIG. 14 shows the artificial hand 100 disposed in the cylindrical grip. Here, the metacarpal-phalangeal joint 504 is locked in a position such that the metacarpal section 508 is disposed at approximately a 30 degree angle with respect to the palm plate 104. Additionally, the thumb 108 is disposed in an open and neutral position. The wrist joint 832 is, oriented such that the palm plate 104 is disposed in a vertical orientation. In this position, the artificial hand 100 is used to grip a flashlight 1404. As can be seen, the phalanges 112 are bent towards the palm plate 104, such that they grip the flashlight 1404. As can be appreciated, the cylindrical grip may be used to grip other similarly shaped objects, such as a drinking glass or a broom handle. It is additionally noted that the diameter of the cylindrical object that is gripped may affect the positioning of the metacarpal-phalangeal joint 504, the phalanges 112, or the thumb 108. For example, in the case of a thicker cylindrical object, the thumb 108 may be rotated past its open position. If this occurs, the tension in the cord 204 will still urge the thumb 108 in a rotation motion towards the palm plate 104, thereby maintaining a pressure against the cylindrical object. Additionally, the metacarpal section 508 may be rotated to a different angle, such as 0 degrees, with respect to the palm plate 104, and the phalanges 112 may be unbent to an appropriate extent. In the case of a thinner cylindrical object, the thumb 108 may be positioned between its open and closed position. If this occurs, the tension the cord 204 will still urge the thumb 108 in a rotation motion towards the palm plate 104, thereby maintaining a pressure against the cylindrical object. Additionally, the metacarpal section 508 may be rotated to a different angle, such as 60 degrees, with respect to the palm plate 104, and the phalanges 112 may be bent or unbent to an appropriate extent.

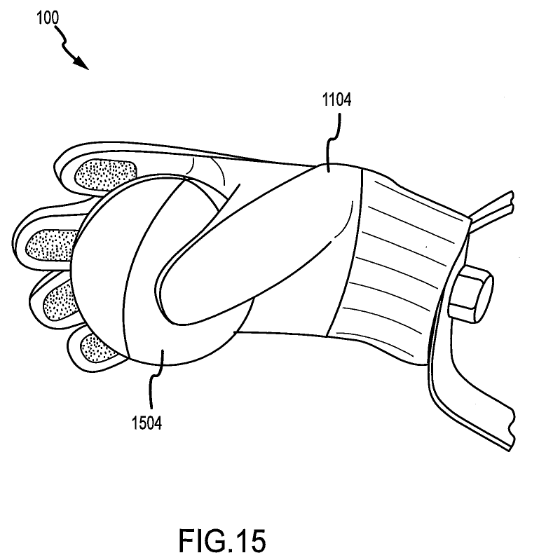

FIG. 15 shows the artificial hand 100 disposed in the spherical grip. Here, the metacarpal-phalangeal joint 504 is locked in a position such that the metacarpal section 508 is disposed at approximately a 30 degree angle with respect to the palm plate 104. Additionally, the thumb 108 is disposed in an open or semi-pronated position. In this position, the artificial hand 100 is used to grip a ball 1504. Here, the phalanges 112 are bent towards palm plate 104 around the ball 1504. Depending on the diameter of the ball 1504, the thumb 108 may be placed in different rotational positions around the U-bolt 124. For at least one ball diameter, the thumb 108 may be positioned between its open and closed position. If this occurs, the tension the cord 204 will urge the thumb 108 in a rotation motion towards the palm plate 104, thereby maintaining a pressure against the ball 1504.

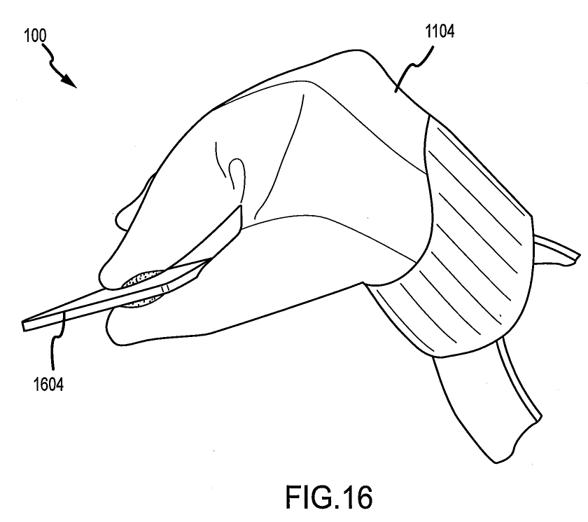

FIG. 16 shows the artificial hand 100 disposed in the tip-pinch grip. Here, the metacarpal-phalangeal joint 504 is locked in a position such that the metacarpal section 508 is disposed at approximately a 60 degree angle with respect to the palm plate 104. Additionally, the thumb 108 is disposed in an open and neutral position. In this position, the artificial hand 100 is used to pinch a card key 1604. Here, the phalanges are unbent or bent to a minor extent such that the tip of the thumb 108 and the tip of at least one phalange 112 meet at a point. In accordance with embodiments of the present invention, the artificial hand 100 may be provided with at least one rigid finger to facilitate pinching objects between the thumb 108 and the phalanges 112.

In accordance with embodiments of the present invention, all the metal parts of the artificial hand 100 are made from the same type of metal. Accordingly, if the artificial hand 100 is submerged in water or brine, no galvanic or electrical currents are established between parts of the hand 100. As a result, corrosion of the various components is prevented.

The present invention, in various embodiments, includes components, methods, processes, systems and/or apparatus substantially as depicted and described herein, including various embodiments, subcombinations, and subsets thereof. Those of skill in the art will understand how to make and use the present invention after understanding the present disclosure. The present invention, in various embodiments, includes providing devices and processes in the absence of items not depicted and/or described herein or in various embodiments hereof, including in the absence of such items as may have been used in previous devices or processes, e.g., for improving performance, achieving ease and/or reducing cost of implementation.

It is to be noted that the term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover, though the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A pre-positionable prosthetic hand, comprising:
a palm plate; and
a thumb interconnected to the palm plate through an attachment member, the attachment member being attached to the thumb at a base of the thumb, wherein the thumb is slidably moveable along the attachment member, and wherein the thumb is rotatable about the attachment member.

2. The prosthetic hand of claim 1, further comprising:
a phalangeal section rotatably attached to the palm plate, wherein the phalangeal section is selectively postionable in a plurality of discrete positions between −90 degrees and +90 degrees with respect to a plane defined by the palm plate.

3. The prosthetic hand of claim 1, further comprising:
a wrist joint providing a movable connection between the palm plate and a base member, the base member being rigidly connected to a bead, wherein an end point of the palm plate is rotatable around the bead.

4. The prosthetic hand of claim 1, further comprising:
a thumb biasing member connected to the palm plate and to the thumb, the thumb biasing member being operable to urge the thumb in at least one of an open position and a closed position.

5. The prosthetic hand of claim 1, wherein the attachment member includes a U-bolt and the base of the thumb includes a U-bolt hole, wherein the U-bolt is received through the U-bolt hole and attached to the palm plate at both ends of the U-bolt.

6. The prosthetic hand of claim 4, wherein the thumb biasing member includes a spring disposed on the dorsal side of the hand, a first end of the spring being attached to palm plate, and a second end of the spring being attached to a cord, the cord being disposed through a cord hole in the palm plate and attached to the thumb.

7. The prosthetic hand of claim 6, wherein the thumb includes a bushing moveable within a bushing track, the bushing being attached to the cord, wherein with the bushing at a first end of the bushing track, the thumb is disposed in the closed position, and with the bushing at a second end of the bushing track, the thumb is disposed in the open position.

8. The prosthetic hand of claim 7, wherein the bushing track includes an arcuate shape having a neutral-bias point, wherein with the bushing between the neutral-bias point and the first end of the bushing track, the thumb biasing member urges the thumb towards the closed position, and with the bushing between the neutral-bias point and the second end of the bushing track, the thumb biasing member urges the thumb towards the open position.

9. The prosthetic hand of claim 8, wherein the neutral bias point is located closer to the second end of the bushing track than the first end of the bushing track.

10. The prosthetic hand of claim 8, wherein the neutral bias point is located at substantially the center of the bushing track.

11. The prosthetic hand of claim 1, wherein the thumb includes an angled tip.

12. The prosthetic hand of claim 2, wherein the phalangeal section includes an axle having a rotatable connection to the palm plate, the first end of the axle being connected to the phalangeal section at a first mounting point, the second end of the axle being connected to the phalangeal section at a second mounting point, the first and second mounting points allowing translational movement of the axle between a locked position and an unlocked position.

13. The prosthetic hand of claim 12, wherein an axle spring urges the axle towards the locked position, wherein with the axle in the locked position the phalangeal section is locked in one of the plurality of discrete positions, wherein a user may urge the axle into an unlocked position by pressing on an end of the axle, and wherein with the axle in the unlocked position the phalangeal section may freely rotate.

14. The prosthetic hand of claim 13, wherein the second end of the axle includes an enlarged portion operable to engage the second mounting point to provide the plurality of discrete positions of the phalangeal section of the hand.

15. The prosthetic hand of claim 3, wherein the wrist joint includes a wrist core provided in association with a wrist cup, the wrist core being rigidly attached to the palm plate and having the endpoint rotatable around the bead, the wrist cup having a curved inner surface operable to engage a rim portion of the wrist core, wherein movement of the rim of the wrist core along the inner surface of the wrist cup defines a range of motion for the palm plate around the bead.

16. A method of using a prepositionable prosthetic hand, comprising:
moving a thumb along an attachment member, wherein the attachment member interconnects the thumb to a palm plate; and
rotating the thumb about the attachment member, wherein rotational movement of the thumb is biased towards at least one of an open position and a closed position.

17. The method of claim 16, further comprising:
moving a phalangeal section into one of a plurality of locked positions, wherein each locked position includes the phalangeal section disposed at a different angle with respect to the palm plate.

18. The method of claim 17, further comprising:
moving the palm plate into an angled position with respect to a base member of the prepositionable hand.

19. The method of claim 18, further comprising:
moving at least one phalange associated with the prepositionable hand into a bent position.

20. The method of claim 17, wherein the prepositionable hand is moved into at least one the following positions:
a lateral grip, wherein the thumb is disposed in a closed pronated position and the phalangeal section is disposed at about a 60 degree angle with respect to the palm plate;
a hook grip, wherein the thumb is disposed in a closed pronated position and the phalangeal section is disposed at about a 90 degree angle with respect to the palm plate;
a cylindrical grip, wherein the thumb is disposed in an open neutral position and the phalangeal section is disposed at about a 0 degree angle with respect to the palm plate;
a spherical grip, wherein the thumb is disposed in an open semi-pronated position and the phalangeal section is disposed at about a 30 degree angle with respect to the palm plate; and
a spherical grip, wherein the thumb is disposed in an open neutral position and the phalangeal section is disposed at about a 30 degree angle with respect to the palm plate.

21. A pre-positionable artificial human hand, comprising:
a thumb;
means for positioning the thumb in a lateral position along an attachment member and in a rotated position around said attachment member; and
means for biasing the thumb in at least one of an open position and a closed position, wherein the open and closed positions are established at rotated positions of the thumb.

22. The prepositionable hand of claim 21, further comprising:
a phalangeal section;
means for adjusting the phalangeal section at a plurality of locked positions, wherein each locked position includes the phalangeal section disposed at different angles with respect to a palm plate that is connected to the means for positioning.

23. The prepositionable hand of claim 22, further comprising:
a base member;
means for positioning the palm at angle with respect to the base member.

24. A pre-positionable prosthetic hand, comprising:
a palm plate;
a thumb interconnected to the palm plate through an attachment member, the attachment member being attached to the thumb at a base of the thumb, wherein the thumb is slidably moveable along the attachment member, and wherein the thumb is rotatable about the attachment member;
a phalangeal section rotatably attached to the palm plate, wherein the phalangeal section is selectively postionable in a plurality of discrete positions between −90 degrees and +90 degrees with respect to a plane defined by the palm plate;
a wrist joint providing a movable connection between the palm plate and a base member, the base member being rigidly connected to a bead, wherein an end point of the palm plate is rotatable around the bead;
a thumb biasing member connected to the palm plate and to the thumb, the thumb biasing member being operable to urge the thumb in at least one of an open position and a closed position;
wherein the attachment member includes a U-bolt and the base of the thumb includes a U-bolt hole, wherein the U-bolt is received through the U-bolt hole and attached to the palm plate at both ends of the U-bolt;
wherein the thumb biasing member includes a spring disposed on the dorsal side of the hand, a first end of the spring being attached to palm plate, and a second end of the spring being attached to a cord, the cord being disposed through a cord hole in the palm plate and attached to the thumb;
wherein the thumb includes a bushing moveable within a bushing track, the bushing being attached to the cord, wherein with the bushing at a first end of the bushing track, the thumb is disposed in the closed position, and with the bushing at a second end of the bushing track, the thumb is disposed in the open position;
wherein the bushing track includes an arcuate shape having a neutral-bias point, wherein with the bushing between the neutral-bias point and the first end of the bushing track, the thumb biasing member urges the thumb towards the closed position, and with the bushing between the neutral-bias point and the second end of the bushing track, the thumb biasing member urges the thumb towards the open position;
wherein the neutral bias point is located closer to the second end of the bushing track than the first end of the bushing track;
wherein the neutral bias point is located at substantially the center of the bushing track;
wherein the thumb includes an angled tip;
wherein the phalangeal section includes an axle having a rotatable connection to the palm plate, the first end of the axle being connected to the phalangeal section at a first mounting point, the second end of the axle being connected to the phalangeal section at a second mounting point, the first and second mounting points allowing translational movement of the axle between a locked position and an unlocked position;
wherein an axle spring urges the axle towards the locked position, wherein with the axle in the locked position the phalangeal section is locked in one of the plurality of discrete positions, wherein a user may urge the axle into an unlocked position by pressing on an end of the axle, and wherein with the axle in the unlocked position the phalangeal section may freely rotate;
wherein the second end of the axle includes an enlarged portion operable to engage the second mounting point to provide the plurality of discrete positions of the phalangeal section of the hand;
wherein the wrist joint includes a wrist core provided in association with a wrist cup, the wrist core being rigidly attached to the palm plate and having the endpoint rotatable around the bead, the wrist cup having a curved inner surface operable to engage a rim portion of the wrist core, wherein movement of the rim of the wrist core along the inner surface of the wrist cup defines a range of motion for the palm plate around the bead.
